(19) United States
(12) Patent Application Publication
(10) Pub. No.: US 2026/0053417 A1
(43) Pub. Date: Feb. 26, 2026

(54) CATHETER DEPTH PERCEPTION TOOLS ON MAP

(71) Applicant: BIOSENSE WEBSTER (ISRAEL) LTD., Yokneam (IL)

(72) Inventors: FADY MASSARWA, BAKA AL GHARBIYYA (IL); MEYTAL SEGEV, HAIFA (IL); AKRAM ZOABI, HAIFA (IL); SIGAL ALTMAN, RAMAT HASHOFET (IL); NAOR AVIDOR MATALON, HAIFA (IL)

(21) Appl. No.: 19/263,573

(22) Filed: Jul. 9, 2025

Related U.S. Application Data
(60) Provisional application No. 63/685,463, filed on Aug. 21, 2024.

(51) Int. Cl. A61B 5/339; A61B 5/00; A61B 5/367; G06T 15/00; G06T 15/06; G06T 15/20; G06T 15/50

(52) U.S. Cl. CPC A61B 5/339; A61B 5/367; A61B 5/6887; G06T 15/005; G06T 15/06; G06T 15/20; G06T 15/506; G06T 2210/41

(57) ABSTRACT

A system includes a display and a processor configured to receive an electroanatomical 3D map of cardiac chamber to be displayed on the display as a view-dependent orthographic projection. The processor is further configured to (i) receive position information of catheter located within the cardiac chamber, from catheter positioning system, (ii) define a reference point location of the catheter displayed on the map, (iii) for any given viewing direction of the map, define the viewing direction as a ray superimposed on a vector from a virtual viewer to the reference point location, (iv) calculate a view-dependent cardiac chamber distance between a distal wall location along the ray and a proximal wall location along the ray, (v) calculate a location over the ray of the reference point location relative to the cardiac chamber view-dependent distance, based on the position information, and (vi) indicate the calculated reference point location distance to user.

### CROSS-REFERENCE TO RELATED APPLICATIONS

This application claims the benefit of U.S. Provisional Patent Application 63/685,463, filed Aug. 21, 2024, whose disclosure is incorporated herein by reference.

### FIELD OF THE DISCLOSURE

This disclosure relates generally to the analysis of electroanatomical (EA) maps, and specifically to a system and method for representing catheter depth on a map.

### BACKGROUND OF THE DISCLOSURE

A 3D EA map of a cardiac chamber is typically presented on a 2D display, which may make it difficult for a physician surveying the map to understand the location of a catheter's distal end assembly relative to the chamber walls.

The present disclosure will be more fully understood from the following detailed description of the examples thereof, taken together with the drawings, in which:

### BRIEF DESCRIPTION OF THE DRAWINGS

FIG. 1 is a schematic, pictorial illustration of a catheter-based electrophysiology (EP) pacing, EA mapping, and ablation system, according to an example of the present disclosure;

FIG. 2 is an EA map (in orthographic view) of a left atrium with catheter depth perception tools incorporated, according to an example of the present disclosure; and

FIG. 3 shows a depth perception achieved with a visualization technique of light and shade applied to a 2D object overlaid on an anatomical map, according to an example of the present disclosure.

### DETAILED DESCRIPTION OF EXAMPLES

### Overview

Cardiac electro-anatomical (EA) catheter mapping is a technique that produces a three-dimensional (3D) EA map of the inner surface of cardiac chamber wall tissue, such as a late activation time (LAT) map of a left atrium. A physician can use the EA map to determine the precise location of an arrhythmia source or, when performing a medical procedure such as cardiac ablation, determine where to apply treatment (e.g., isolation).

A 3D EA map of a cardiac chamber is typically presented on a 2D display as an orthographic projection. Orthographic projection gives an accurate rendering of the cardiac chamber and devices (e.g., catheters) inside. A physician may rotate the map on the display and view the map from any viewing direction in space.

Orthographic projection, however, provides no sense of depth as the distance from the virtual viewer to an object does not affect the size of the rendered object. As a result, a physician viewing the EA map on a display may find it difficult to understand the current location of a catheter's distal end assembly relative to the chamber walls.

Examples of the present disclosure described herein provide catheter depth perception tools that let the physician utilize the advantages of an orthographically projected map together with being given a clear visual indication of the catheter's distal end assembly relative to the chamber walls at any given viewing direction.

In this disclosure, a processor defines a viewing direction of the 3D map as a ray superimposed on a vector from a virtual viewer to a catheter reference point location (e.g., to a catheter's landmark location such as a distal edge location of a shaft of the catheter). The processor receives the position information of the catheter located within the cardiac chamber, from a catheter positioning system.

Any given viewing direction of the 3D map (i.e., the direction of the orthograph projection) defines this way a ray that crosses the near/proximal (e.g., front-side) wall tissue and the remote/distal (e.g., back-side) wall tissue of the cardiac chamber. The crossing locations are defined per that view, respectively, as a proximal point location and a distal point location. The unique ray of the virtual viewer also crosses a catheter reference point location. The processor calculates the location over the ray of the reference point relative to the cardiac chamber view-dependent distance, based on the position information.

This way, the catheter's reference location (e.g., in the depth dimension) can be presented (e.g., on a scale) relative to the proximal wall and distal wall point locations.

In one example, the disclosed technique provides a view-dependent tool on the displayed map comprising a scale of catheter depth that is visualized in two ways:
1. A min-max dimension scale “per given view” near a transparent additional optional view (also called herein auxiliary map) that can be adjusted to a superior view.
2. Visualization overlaid of the catheter's shaft, of its location (e.g., color bar automatically adjusted based on proximity to the proximal point location).

These two visualizations provide a physician real-time sense of the depth, with the actual relative depth value on the scale of the chosen view.

In an example, for any given view direction, a processor presents an auxiliary orthographic view of the EA map (i.e., presents an auxiliary map) as seen from a direction orthogonal to the ray's direction (e.g., a superior view). The auxiliary map shows the catheter's location relative to the distal and proximal point locations along the viewing direction.

In addition, a scale located by the side of the auxiliary map gives the estimated distance of the catheter from the proximal wall relative to the total distance between the distal and proximal walls.

A similar scale can be superimposed on the catheter's shaft so that the physician can focus on the catheter and still receive the value of its depth in the chamber.

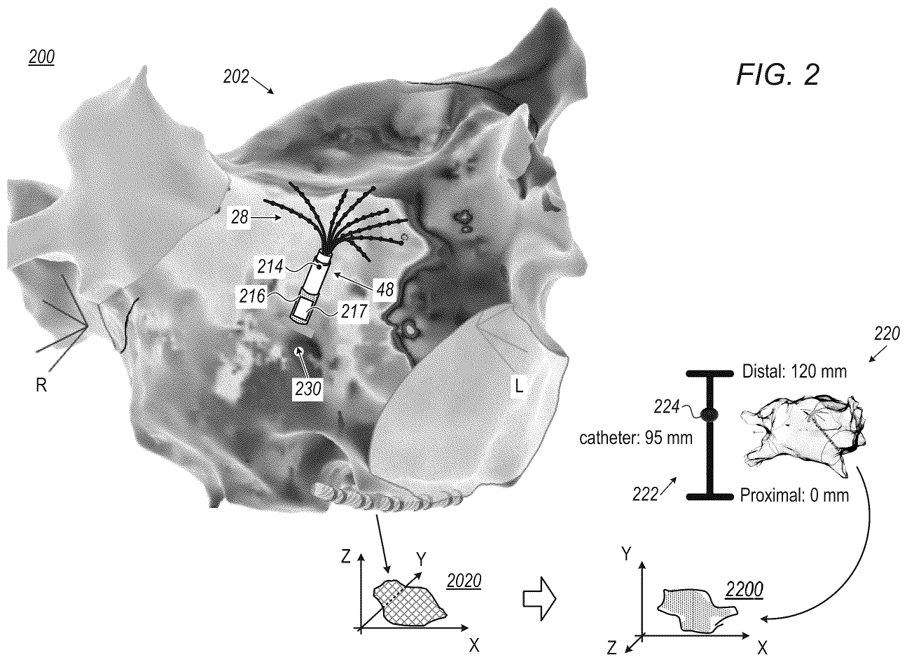

Further to that, the catheter's relative depth is indicated by being graphically encoded; for example, a distal portion of the shaft's color may vary from one color to another depending on how close the catheter is to the proximal and distal point locations. In a specific implementation seen in FIG. 2, the red portion appears larger as the catheter is closer to the distal wall (along the chamber proximal-distal wall distance defined by the viewing ray) and the green portion appears larger as it nears the proximal wall.

In another example, the technique provides depth perception for catheter navigation using light and shadow effects. A processor runs an algorithm that manipulates the catheter's perceived illumination such that the catheter's appearance changes based on its proximity to the viewer. Specifically, the catheter appears to be more illuminated when it is closer (i.e., proximal) to the viewer and more shaded when it is further away (i.e., distal). This visual cue gives the user a sense of the catheter's depth.

In yet another example, a processor colors the catheter image itself or a legend attached to the catheter. The catheter or the attached legend may be colored in one way (e.g., red) if the catheter is closer to the distal wall, while colored in another way (e.g., green) if the catheter is closer to the proximal wall.

Finally, the disclosed tool can also be applied or assist in veins anatomy perception to enhance optimization of ablating catheter location to improve electrical ablation.

The disclosed technique allows a physician to easily and intuitively view and comprehend the catheter's location in the depth dimension in the 3D maps using several unique visual presentations.

### System Description

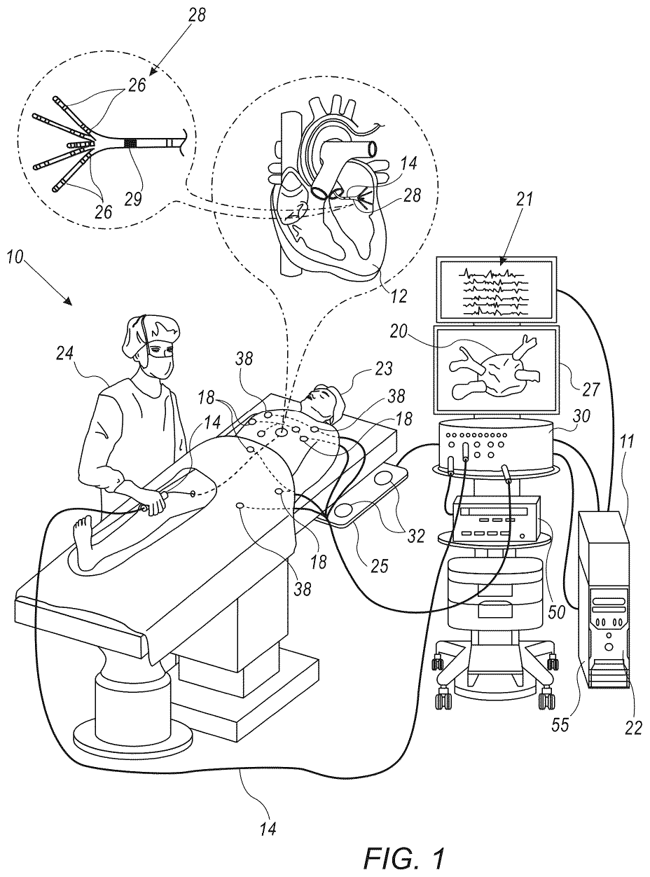

FIG. 1 is a schematic, pictorial illustration of a catheter-based electrophysiology (EP) pacing, EA mapping, and ablation system 10, according to an example of the present disclosure.

System 10 includes multiple catheters, which are percutaneously inserted by a physician 24 through the patient's vascular system into a chamber or vascular structure of a heart 12. Typically, a delivery sheath catheter is inserted into the left or right atrium near a desired location in heart 12. Thereafter, a plurality of catheters can be inserted into the delivery sheath catheter so as to arrive at the desired location. The plurality of catheters may include catheters dedicated to sensing Intracardiac Electrogram (IEGM) signals and/or mapping heart 12, catheters dedicated to ablating, and/or catheters dedicated to both sensing and/or mapping and ablating. An example catheter 14 that is configured for sensing and mapping is illustrated herein. Physician 24 brings a distal end assembly 28 of catheter 14 into contact with the heart wall for sensing electro-potentials at a target site in heart 12, as well as for mapping one or more chambers of the heart, such as the left or right atriums.

Catheter 14 is an exemplary multi-spine catheter that includes multiple electrodes 26 distributed on the spines of the catheter. Catheter 14 may additionally include a position sensor 29 embedded in or near distal end assembly 28 for tracking the position and orientation of distal end assembly 28. Optionally and preferably, position sensor 29 is a magnetic based position sensor including three magnetic coils for sensing three-dimensional (3D) position and orientation.

A magnetic-based position sensor 29 may be operated together with a location pad 25, including a plurality of magnetic coils 32 configured to generate magnetic fields in a predefined working volume. The real-time position of distal end assembly 28 of catheter 14 may be tracked based on magnetic fields generated with location pad 25 and sensed by magnetic-based position sensor 29. Details of the magnetic-based position sensing technology are described in U.S. Pat. Nos. 5,5391, 199; 5,443,489; 5,558,091; 6,172,499; 6,239, 724; 6,332,089; 6,484,118; 6,618, 612; 6,690,963; 6,788,967; 6,892,091.

System 10 includes one or more electrode patches 38 positioned for skin contact on a patient 23 to establish a location reference for location pad 25 as well as impedance-based tracking of at least some electrodes 26. For impedance-based tracking, electrical current is directed toward electrodes 26 and sensed at electrode skin patches 38 so that the location of each electrode can be triangulated via the electrode patches 38. Details of the impedance-based location tracking technology are described in U.S. Pat. Nos. 7,536,218; 7, 756, 576; 7,848, 787; 7,869,865; and 8,456,182.

A recorder 11 displays electrograms 21 captured with body surface ECG electrodes 18 and intracardiac electrograms (IEGM) that may be captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to one or more of electrodes 26. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Patient interface unit (PIU) 30 is an interface configured to establish electrical communication between catheters, electrophysiological equipment, a power supply and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, a processor 22 with memory or storage with appropriate operating software loaded therein, and user interface capability. Workstation 55 may provide multiple functions, optionally including (1) modeling the endocardial anatomy in three-dimensions (3D) and rendering a model or anatomical map 20 of all or part of heart 12 for display on a display device 27, typically by first mapping points of a selected portion of the heart, forming a mesh of the points, then covering the mesh with a surface, (2) displaying on display device 27 activation sequences (or other data) compiled from recorded electrograms 21 in representative visual indicia or imagery superimposed on the rendered anatomical map 20, (3) displaying real-time location and orientation of distal end assembly 28 within the heart chamber, and (4) displaying on display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

In general, processor 22 may be embodied as a single processor, or as a cooperatively networked or clustered set of processors. The functionality of the processor may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, the processor may be embodied as a programmed processor comprising, for example, a central processing unit (CPU) and/or a Graphics Processing Unit (GPU). Program code, including software programs and/or data, may be loaded for execution and processing by the CPU and/or GPU. The program code and/or data may be downloaded to the processor electronically, over a network, for example. Alternatively, or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

### Catheter Depth Perception Tools on Map

FIG. 2 is an EA map 200 (viewed as an orthographic projection 202) of a left atrium with catheter depth perception tools (220, 222, 230) incorporated, according to an example of the present disclosure. FIG. 2 shows a multi-spine, multi-electrode, distal end assembly 28 catheter inside the cardiac chamber. The physician cannot know how posterior or anterior the multi-electrode assembly 28 is in any given view.

As many modern catheter assemblies (e.g., multi-spine, basket, lasso) have extended shapes over space, trying to indicate the location of individual elements (e.g., electrodes, spines) of the assembly may result in visual clutter.

To mitigate this, the disclosed technique defines a catheter reference point location to represent the assembly. One such reference point location can be location 214 on the distal edge of the catheter shaft 48. Another reference point location may be the center of a volume of revolution defined by the assembly (e.g., the center of an inner volume of a basket). The position tracking system continuously calculates the catheter's location during the EA mapping, as described in FIG. 1, thereby making the reference point location value known per each view.

The disclosed technique provides a distance scale of 222 of catheter depth that shows a representation (224) of the catheter's location 214 along the distance scale 222, relative to the cardiac chamber's view-dependent distal and proximal wall point locations.

To ease the physician's workflow, the catheter's view-dependent depth location 214 is also shown (230) near shaft 48. The current catheter position of 95 mm over a 120 mm view-dependent distance 222 between the cardiac walls is much more distal than proximal. To visually reflect this distal position, the disclosed technique colors (217) a catheter shaft 48 portion 216 near assembly 28 in a first color (e.g., red).

If the operator moves the catheter proximally so that the catheter's location 214 value becomes smaller than 60 mm, portion 216 (e.g., bar 216's) color 217 will change to a second color (e.g., green).

In the shown example, the processor presents a view-dependent auxiliary orthographic view 220 of map 200 from a superior view. Transparent auxiliary map 220 is received as an orthographic projection of the 3D EA map from a 90-degree rotated X-Y view 2200 of the X-Z view 2020 of orthographic projection 202 of map 202. Auxiliary map 220 shows the catheter's location relative to the distal and proximal cardiac chamber walls along the original viewing direction used in orthographic projection 202 of map 200.

### Catheter Depth Perception by Shading

As noted above, the disclosed technique may provide depth perception for catheter navigation using light and shadow effects. In this example, a processor runs an algorithm that manipulates perceived lighting on the catheter such that the catheter's image appearance changes based on its proximity to the viewer. Specifically, the catheter appears to be more illuminated when it is closer (i.e., proximal) to the viewer and darker when it is farther away (i.e., distal). This visual cue may give the user a sense of the catheter's depth.

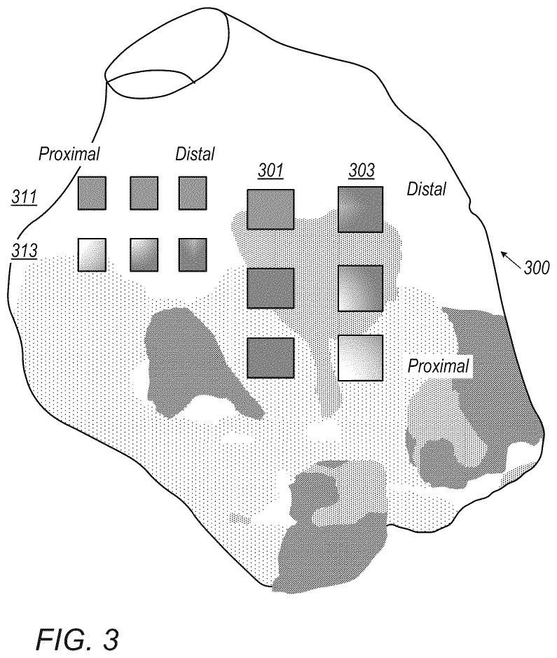

FIG. 3 shows a depth perception achieved with a visualization technique of light and shade applied to a 2D object overlaid on an anatomical map, according to an example of the present disclosure.

As seen in column 301 and row 311 of the 2D object, no sense of depth is achieved wherever the depth of the 2D object is assumed to be (i.e., from proximally to distally as seen by virtual viewer).

Column 303 and row 313, on the other hand, demonstrate how manipulating the light and shade of the 2D object visualization yields a sense of the object's depth. For example, if the 2D object is overlayed on a catheter, the catheter would appear to be more illuminated when it is closer to the viewer and appear more shaded when it is farther away. In other words, the illumination and shading of the 2D object would be determined by a virtual light-source (not shown) placed above the portion of the 3D map more proximal to the viewer.

The shaded 2D object may be a legend attached to the catheter or be the catheter image itself.

### EXAMPLES

### Example 1

A system (10) includes a display device (27) and a processor (22) configured to receive an electroanatomical (EA) 3D map (20, 200) of a cardiac chamber, the map configured to be displayed on the display device as a view-dependent (2200, 2020) orthographic projection (202). The processor (22) is further configured to (i) receive position information of a catheter (14) located within the cardiac chamber, from a catheter positioning system, (ii) define a reference point location (214) of the catheter displayed on the 3D map, (iii) for any given viewing direction of the 3D map, define the viewing direction as a ray superimposed on a vector from a virtual viewer to the reference point location, (iv) calculate a view-dependent cardiac chamber distance (222) between a distal wall location along the ray and a proximal wall location along the ray, (v) calculate a location over the ray of the reference point location (214) relative to the cardiac chamber view-dependent distance (222), based on the position information, (vi) indicate (217, 224, 303) the calculated reference point location (214) relative to the cardiac chamber view-dependent distance (222) to a user.

### Example 2

The system (10) according to example 1, wherein the processor (22) is configured to indicate the calculated reference point location (214) by displaying (224) the calculated location value over a numerical scale defined by the cardiac chamber view-dependent (2200, 2020) distance (222).

### Example 3

The system (10) according to any of examples 1 and 2, wherein the processor (22) is configured to indicate the calculated reference point location (214) by displaying an auxiliary map (220) obtained from an orthographic view from a direction orthogonal to the given viewing direction.

### Example 4

The system (10) according to any of examples 1 through 3, wherein the processor (22) is configured to indicate the calculated reference point location (214) by graphically encoding (217) a portion (216) of the catheter (14) according to the calculated reference location value (224) over a numerical scale defined by the cardiac chamber view-dependent distance (222).

### Example 5

The system (10) according to any of examples 1 through 4, wherein the processor (22) is configured to graphically encode (217) the portion (216) of the catheter (14) by a first color if the calculated reference location value is smaller than half the cardiac chamber view-dependent distance (222) and by a second color if the calculated reference location value is higher than half the cardiac chamber view-dependent distance (222).

### Example 6

The system (10) according to any of examples 1 and 5, wherein the processor (22) is configured to indicate the calculated reference point location (214) by displaying at least a portion (28, 48) of the catheter (14) as illuminated differently (303, 313) according to its view-dependent distance (224) from the user.

### Example 7

The system (10) according to any of examples 1 and 5, wherein the processor (22) is configured to display at least a portion (28, 48) of the catheter (14) as illuminated differently by making the catheter appear (303, 313) to be more illuminated when it is closer to the viewer and appear more shaded when it is further away.

### Example 8

A method includes receiving an electroanatomical (EA) 3D map (20, 200) of a cardiac chamber, the map configured to be displayed on a display device (27) as a view-dependent orthographic projection. Position information is received, of a catheter located within the cardiac chamber, from a catheter positioning system. A reference point location (214) is defined, of the catheter displayed on the 3D map. For any given viewing direction of the 3D map, the viewing direction is defined as a ray superimposed on a vector from a virtual viewer to the reference point location (214). A view-dependent cardiac chamber distance (222) is calculated, between a distal wall location along the ray and a proximal wall location along the ray. A location is calculated, over the ray of the reference point location relative to the cardiac chamber view-dependent distance, based on the position information. The calculated reference point location (214) relative to the cardiac chamber view-dependent distance is indicated (217, 224, 303) to a user.

It will be appreciated that the examples described above are cited by way of example and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A system, comprising:
a display device; and
a processor, which is configured to:
receive an electroanatomical (EA) 3D map of a cardiac chamber, the map configured to be displayed on the display device as a view-dependent orthographic projection;
receive position information of a catheter located within the cardiac chamber, from a catheter positioning system;
define a reference point location of the catheter displayed on the 3D map;
for any given viewing direction of the 3D map, define the viewing direction as a ray superimposed on a vector from a virtual viewer to the reference point location;
calculate a view-dependent cardiac chamber distance between a distal wall location along the ray and a proximal wall location along the ray;
calculate a location over the ray of the reference point location relative to the cardiac chamber view-dependent distance, based on the position information; and
indicate the calculated reference point location relative to the cardiac chamber view-dependent distance to a user.

2. The system according to claim 1, wherein the processor is configured to indicate the calculated reference point location by displaying the calculated location value over a numerical scale defined by the cardiac chamber view-dependent distance.

1. The system according to claim 1, wherein the processor is configured to indicate the calculated reference point location by displaying an auxiliary map obtained from an orthographic view from a direction orthogonal to the given viewing direction.

4. The system according to claim 1, wherein the processor is configured to indicate the calculated reference point location by graphically encoding a portion of the catheter according to the calculated reference location value over a numerical scale defined by the cardiac chamber view-dependent distance.

5. The system according to claim 4, wherein the processor is configured to graphically encode the portion of the catheter by a first color if the calculated reference location value is smaller than half the cardiac chamber view-dependent distance and by a second color if the calculated reference location value is higher than half the cardiac chamber view-dependent distance.

6. The system according to claim 1, wherein the processor is configured to indicate the calculated reference point location by displaying at least a portion of the catheter as illuminated differently according to its view-dependent distance from the user.

7. The system according to claim 1, wherein the processor is configured to display at least a portion of the catheter as illuminated differently by making the catheter appear to be more illuminated when it is closer to the viewer and appear more shaded when it is further away.

8. A method, comprising:
receiving an electroanatomical (EA) 3D map of a cardiac chamber, the map configured to be displayed on a display device as a view-dependent orthographic projection;
receiving position information of a catheter located within the cardiac chamber, from a catheter positioning system;
defining a reference point location of the catheter displayed on the 3D map;
for any given viewing direction of the 3D map, defining the viewing direction as a ray superimposed on a vector from a virtual viewer to the reference point location;
calculating a view-dependent cardiac chamber distance between a distal wall location along the ray and a proximal wall location along the ray;
calculating a location over the ray of the reference point location relative to the cardiac chamber view-dependent distance, based on the position information; and
indicating the calculated reference point location relative to the cardiac chamber view-dependent distance to a user.

9. The method according to claim 8, wherein indicating the calculated reference point location comprises displaying the calculated location value over a numerical scale defined by the cardiac chamber view-dependent distance.

10. The method according to claim 8, wherein indicating the calculated reference point location comprises displaying an auxiliary map obtained from an orthographic view from a direction orthogonal to the given viewing direction.

11. The method according to claim 8, wherein indicating the calculated reference point location comprises graphically encoding a portion of the catheter according to the calculated reference location value over a numerical scale defined by the cardiac chamber view-dependent distance.

12. The method according to claim 11, and comprising graphically encoding the portion of the catheter by a first color if the calculated reference location value is smaller than half the cardiac chamber view-dependent distance and by a second color if the calculated reference location value is higher than half the cardiac chamber view-dependent distance.

13. The method according to claim 8, wherein indicating the calculated reference point location comprises displaying at least a portion of the catheter as illuminated differently according to its view-dependent distance from the user.

14. The method according to claim 8, and comprising displaying at least a portion of the catheter as illuminated differently by making the catheter appear to be more illuminated when it is closer to the viewer and appear more shaded when it is further away.
